(19) United States
(12) Patent Application Publication
(10) Pub. No.: US 2009/0089899 A1
(43) Pub. Date: Apr. 2, 2009

(54) Method for Enhancing Drought Stress Tolerance in Plants by Active AREB1

(76) Inventors: Kazuko Shinozaki, Ibaraki (JP); Yasunari Fujita, Ibaraki (JP); Kyonoshin Maruyama, Ibaraki (JP)

Correspondence Address: Davidson, Davidson & Kappel, LLC, 485 7th Avenue, 14th Floor, New York, NY, 10018, US

(21) Appl. No.: 12/084,346

(22) PCT Filed: Mar. 15, 2006
(86) PCT No.: PCT/JP2006/305634

§ 371 Date: Apr. 29, 2008

(30) Foreign Application Priority Data
Nov. 1, 2005 (JP) 2005-318871

(51) Int. Cl. C12N 15/29; C12N 15/82

(52) U.S. Cl. 800/289; 536/23.6; 435/320.1; 800/298; 800/278

(57) ABSTRACT

Provided are: active AREB1 capable of improving plant tolerance against environmental stresses such as drought, salt, and low temperature through expression at high levels in plant bodies; and a method for improving plant environmental stress tolerance using the active AREB1. Also provided is an active environmental stress responsive transcription factor AREB1 gene lacking the activation control region of the plant environmental stress responsive transcription factor AREB1 and containing an N-terminal transcriptional activation domain and a DNA binding domain.

### TECHNICAL FIELD

The present invention relates to an active AREB1 protein in its activated form that controls drought stress tolerance and a method for using the protein.

### BACKGROUND ART

Water deficiency results in fatal damage to all forms of life. Hence, plants have developed the complicated signaling network mechanisms to cope with environmental stresses such as drought stress, so as to avoid such fatal damage.

A plant hormone, abscisic acid (ABA), has a variety of functions in plants and is also involved in adaptive response to environmental stresses. Abscisic acid is synthesized under water-deficient conditions and plays an important role in responses to drought stress. Many genes that are induced by drought stress are known, and many of them are activated by abscisic acid. ABRE (ABA-responsive element) is known as a promoter for genes that are controlled by such abscisic acid.

Regarding AREB, AREB1, AREB2, and AREB3 of Arabidopsis thaliana have been reported for the first time as proteins that bind to ABRE (see Uno et al., Proc. Natl. Acad. Sci. U.S.A. 97, 11632-11637 (2000)). Nine AREB homologs including AREB1/ABF2, AREB2/ABF4, AREB3/DPBF3, ABF1, ABF3/DPBF5, ABI5/DPBF1, EEL/DPBF4, DPBF2, and AT5G42910 of Arabidopsis thaliana have been reported to date (see Uno et al., Proc. Natl. Acad. Sci. U.S.A. 97, 11632-11637 (2000); Choi et al., J, Biol. Chem. 275, 1723-1730 (2000); Finkelstein et al., Plant Cell 12, 599-609 (2000); Lopez-Molina et al., Plant Cell Physiol. 41, 541-547 (2000); Bensmihen et al., Plant Cell 14, 1391-1403 (2002); Jakoby et al., Trends plant Sci. 7, 106-111 (2002); Kim et al., Plant Physiol. 130, 688-697 (2002); and Suzuki et al., Plant Physiol. 132, 1664-1677 (2003)). AREB is a control factor that transcribes gene expression through mediation of ABA. AREB is known as a factor for controlling drought stress tolerance, salt stress tolerance, and low-temperature stress tolerance genes. AREB has functions equivalent to those of DREB for the control of drought stress tolerance, salt stress tolerance, and low-temperature stress tolerance. AREB1 binds to ABRE so as to induce the expression of a stress responsive gene downstream thereof, thereby imparting drought stress, salt stress, and low-temperature stress responsiveness to plants. High-level expression of AREB1 alone within a plant body does not induce the expression of such stress responsive gene downstream thereof. It is thought that AREB1 activation requires stress-induced splicing and the phosphorylation of the AREB1 protein.

### DISCLOSURE OF THE INVENTION

An object of the present invention is to provide active AREB1 that enhances drought stress tolerance, salt stress tolerance, and low-temperature stress tolerance in plants via its high-level expression within the plant bodies and to provide a method for enhancing drought stress tolerance, salt stress tolerance, and low-temperature stress tolerance in plants using such active AREB1.

The present inventors have conducted detailed domain analysis of AREB1, so as to specify that an AREB1 transcriptional activation domain is a region of the N-terminal amino acids 1 to 60. Furthermore, the present inventors have discovered that AREB1 is altered to be in a constantly activated form when an activation control region (amino acids 65-277) between the transcriptional activation domain and a DNA binding domain is deleted. Microarray analysis has been conducted using Arabidopsis thaliana prepared via introduction of such active AREB1. High-level expression of various stress tolerance genes has thus been demonstrated as a result of analysis of target genes of AREB1. Moreover, Arabidopsis thaliana prepared via introduction of active AREB1 has been subjected to a stress tolerance test, so that enhanced drought stress tolerance has been confirmed. Furthermore, it has been identified that, based on phylogenetic tree analysis, AREB2 and ABF3 of Arabidopsis thaliana, which are AREB1 homologs, are drought stress, salt stress, and low-temperature stress responsive genes that are mediated by ABA signals. Furthermore, the present inventors have succeeded in identification of a drought stress, salt stress, and low-temperature stress responsive homologous gene OsAREB1 not only in Arabidopsis thaliana, which is a dicotyledon, but also in rice, which is a monocotyledon. The N-terminal region specified herein is an extremely highly homologous region regardless of plant species. Hence, it is concluded that techniques used for the production of active AREB1 are applicable broadly to a variety of plant species.

The present invention is as described below.

[1] An active environmental stress responsive transcription factor AREB1 gene, lacking a part or the whole of an activation control region of a plant environmental stress responsive transcription factor AREB1 and containing the N-terminal transcriptional activation domain and a DNA binding domain.
[2] An active environmental stress responsive transcription factor AREB1 gene, lacking at least one of the Q, R, S, and T regions of a plant environmental stress responsive transcription factor and containing a P region that contains the N-terminal transcriptional activation domain and an U region that contains a DNA binding domain.
[3] The active environmental stress responsive transcription factor AREB1 gene according to [1] or [2], which is derived from Arabidopsis thaliana.
[4] The active environmental stress responsive transcription factor AREB1 gene according to [3], encoding a protein that comprises an amino acid sequence derived from the amino acid sequence represented by SEQ ID NO: 2 by deletion of an amino acid sequence ranging from amino acids 65 to 277.
[5] An active environmental stress responsive transcription factor AREB1 gene, encoding a protein that comprises an amino acid sequence derived from the amino acid sequence of the active environmental stress responsive transcription factor AREB1 gene according to [3] or [4] by deletion, substitution, or addition of one or several amino acids and has plant environmental stress responsive transcriptional activity.
[6] The active environmental stress responsive transcription factor AREB1 gene according to [3], comprising a nucleotide sequence derived from the nucleotide sequence represented by SEQ ID NO: 1 by deletion of a nucleotide sequence ranging from nucleotides at positions 312 to 950.
[7] An active environmental stress responsive transcription factor AREB1 gene, comprising DNA that is capable of hybridizing under stringent conditions to DNA comprising a nucleotide sequence complementary to DNA comprising the nucleotide sequence of the active environmental stress responsive transcription factor AREB1 gene according to [6] and encodes a protein having plant environmental stress responsive transcriptional activity.
[8] An active environmental stress responsive transcription factor AREB1 gene, lacking an activation control region of an environmental stress responsive transcription factor OsAREB1 derived from rice and containing a transcriptional activation domain and a DNA binding domain.
[9] The active environmental stress responsive transcription factor AREB1 gene according to any one of [1] to [8], in which the environmental stress is drought stress, salt stress, or low-temperature stress.
[10] A plant transformation vector, containing the gene according to any one of [1] to [9].
[11] A transgenic plant, having enhanced environmental stress tolerance as a result of transformation with the plant transformation vector according to [10].
[12] A method for enhancing the environmental stress tolerance of a plant via introduction of the gene according to any one of [1] to [9] into the plant.
[13] The method for enhancing environmental stress tolerance according to [12], in which the environmental stress tolerance is drought tolerance, salt tolerance, and/or low-temperature tolerance.

This description includes part or all of the contents as disclosed in the description and/or drawings of Japanese Patent Application No. 2005-318871, which is a priority document of the present application.

### BRIEF DESCRIPTION OF THE DRAWINGS

FIG. 1A is a figure relating to the domain analysis of AREB1 using protoplasts of Arabidopsis thaliana T87 cells, and it specifically shows the constructs of an effector plasmid and a reporter plasmid.

FIG. 1B is a figure relating to the domain analysis of AREB1 using protoplasts of Arabidopsis thaliana T87 cells, and it specifically shows the results of transactivation domain analysis of AREB1 from which the N-terminus has been deleted.

FIG. 1C is a figure relating to the domain analysis of AREB1 using protoplasts of Arabidopsis thaliana T87 cells, and it specifically shows the results of transactivation of AREB1ΔQT and AREB1ΔP/RT.

FIG. 2A shows photographs showing the results of Northern analysis of active AREB1-expressing Arabidopsis thaliana.

FIG. 2B shows photographs showing the growth of active AREB1-expressing Arabidopsis thaliana in GMK medium.

FIG. 3 shows photographs showing the results of a drought stress tolerance test conducted for active AREB1-expressing Arabidopsis thaliana.

FIG. 4A shows the results of the analysis of the phylogenetic tree of the AREB1 homologous genes in Arabidopsis thaliana and rice. The relationship between the AREB1 homologous gene of Arabidopsis thaliana (dicotyledon) and the AREB1 homologous gene of rice (monocotyledon) is shown with the use of a phylogenetic tree.

FIG. 4B shows photographs showing the gene expression profiles of the AREB1 homologous gene of Arabidopsis thaliana when treatment with ABA, drought, salt, and water was performed.

FIG. 4C shows the gene expression profiles of the AREB1 homologous gene of rice when treatment with drought, salt, and low temperatures was performed.

FIG. 4D shows a comparison of Arabidopsis thaliana with rice in terms of the amino acid sequence of the transcriptional activation region in the AREB1 homologous gene.

### BEST MODE FOR CARRYING OUT THE INVENTION

### 1. Gene of the Present Invention

An active environmental stress responsive transcription factor AREB1 gene lacking a part or the whole of an activation control region of a plant environmental stress responsive transcription factor AREB1 and containing the N-terminal transcriptional activation domain and a DNA binding domain can be prepared by deletion of the activation control region of a gene encoding plant AREB1. Here, the environmental stress refers to drought stress, salt stress, low-temperature stress, or the like.

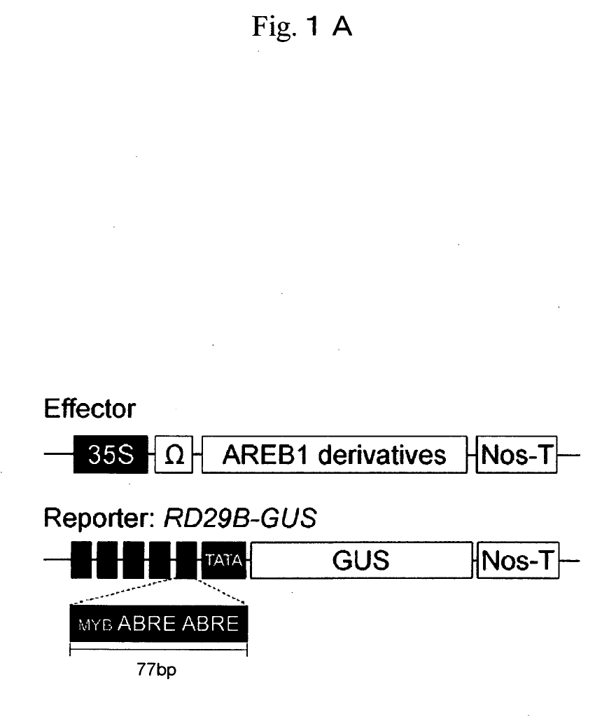

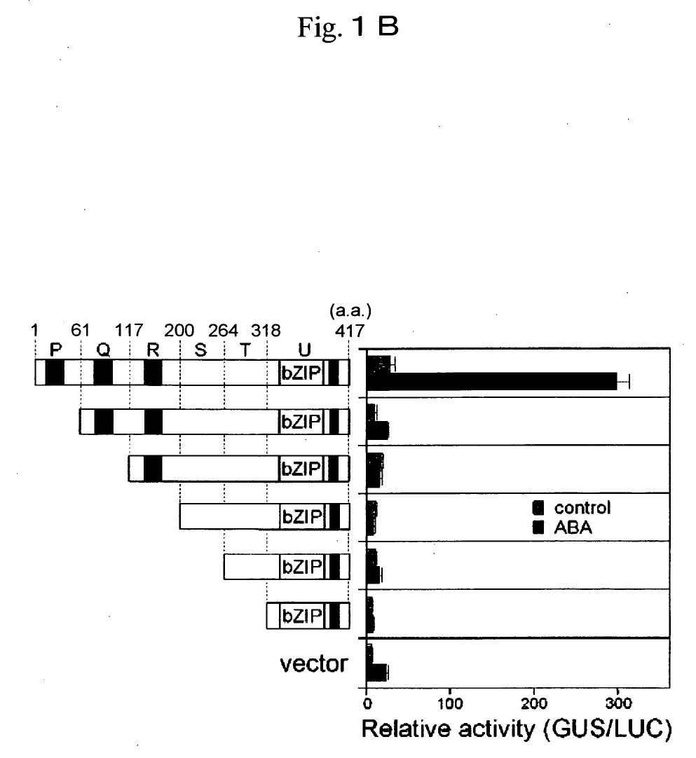

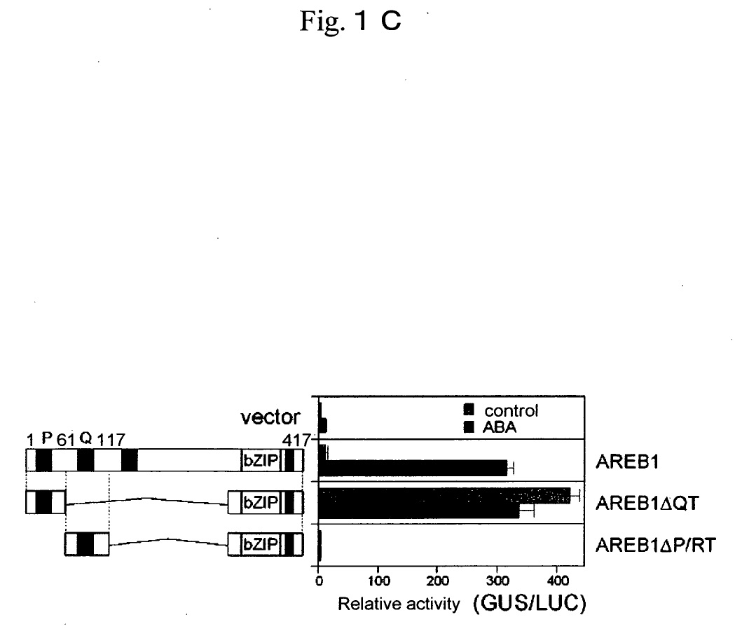

The plant AREB1 gene can be divided into 6 regions: P (amino acids 1 to approximately 60 of the AREB1 protein), Q (approximately amino acids 61 to 116 of the AREB1 protein), R (approximately amino acids 117 to 199 of the AREB1 protein), S (approximately amino acid 200 to 263 of the AREB1 protein), T (approximately amino acids 264 to 317 of the AREB1 protein), and U (approximately amino acids 318 to 417 of the AREB1 protein), as shown in FIG. 1B. Of these, the U region contains bZIP that is a DNA binding domain and the P region contains the transcriptional activation domain. The Q, R, S, and T regions contain the activation control region.

The gene of the present invention contains at least the transcriptional activation domain of the P region and the DNA binding domain of the U region among them. The gene lacks a portion or the whole of the activation control region of the Q, R, S, and T regions and lacks the functions of the activation control region. In the gene of the present invention, the transcriptional activity region containing the transcriptional activation domain has the functions. An example of such a deleted region is at least one of the above Q, R, S, and T regions. Further examples of the same include a deletion ranging from Q to T and a deletion ranging from R to T. Deletion of the whole of each region is not always indispensable. Deletion of a portion of each region is acceptable, as long as the activation control function is lost. An example of the gene of the present invention is a gene encoding a protein that lacks an amino acid sequence comprising sequential 100, 150, 200, 250, 300, or 350 amino acids starting from amino acids 50 to 100, amino acids 61 to 70, or amino acids 100 to 130 among amino acids 61 to 316 of the amino acid sequence represented by SEQ ID NO: 2 of Arabidopsis-thaliana-derived AREB1 protein.

Examples of such gene include:
a gene encoding a protein that comprises an amino acid sequence derived from the amino acid sequence represented by SEQ ID NO: 2 by deletion of an amino acid sequence ranging from any one of amino acids 60 to 70 to any one of amino acids 270 to 280;
a gene encoding a protein that comprises an amino acid sequence derived from the same by deletion of an amino acid sequence ranging from any one of amino acids 60 to 100 to any one of amino acids 260 to 320; and
a gene encoding a protein that comprises an amino acid sequence derived from the same by deletion of an amino acid sequence ranging from any one of amino acids 100 to 130 to any one of amino acids 260 to 320. A preferable example herein is a gene encoding a protein that comprises an amino acid sequence derived from the same by deletion of an amino acid sequence ranging from amino acids 65 to 277.

Furthermore, the gene of the present invention is a gene comprising a nucleotide sequence derived from the nucleotide sequence represented by SEQ ID NO: 1 of the AREB1 gene of Arabidopsis thaliana by deletion of sequential 300, 450, 600, 750, 900, or 1050 nucleotides starting from nucleotides at positions 270 to 420, nucleotides at positions 300 to 330, or nucleotides at positions 420 to 510.

An example of such gene is:
a gene comprising a nucleotide sequence derived from the nucleotide sequence represented by SEQ ID NO: 1 by deletion of a nucleotide sequence ranging from any one of nucleotides at positions 300 to 330 to any one of nucleotides at positions 920 to 960;
a gene comprising a nucleotide sequence derived from the same by deletion of a nucleotide sequence ranging from any one of nucleotides at positions 300 to 420 to any one of nucleotides at positions 1000 to 1080; or
a gene comprising a nucleotide sequence derived from the same by deletion of a nucleotide sequence ranging from any one of nucleotides at positions 420 to 510 to any one of nucleotides at positions 1000 to 1080. A preferable example herein is a gene that comprises a nucleotide sequence derived from the same by deletion of a sequence ranging from nucleotides at positions 312 to 950.

The above described gene lacking a portion or the whole of the activation control region can be prepared by known gene engineering techniques. For example, DNA encoding a transcriptional activation domain may be ligated to DNA encoding a DNA binding domain using PCR (Innis et al., 1990, PCR Protocols, Academic Press, San Diego). Moreover, both DNAs can also be ligated by a mutual priming method (Uhlmann, 1988, Gene 71: 29-40).

In the present invention, AREB1 lacking a portion or the whole of the activation control region of the plant environmental stress responsive transcription factor AREB1 and containing the N-terminal transcriptional activation domain and a DNA binding domain is referred as active AREB1. Moreover, AREB1 lacking the above Q-to-T regions is referred to as AREB1ΔQT, for example.

Another example of the gene of the present invention is a gene encoding a protein that comprises an amino acid sequence derived from the above amino acid sequence by deletion, substitution, or addition of one or several amino acids and has plant environmental stress responsive transcriptional activity. Here, “several (amino acids)” means 20 or less, preferably 10 or less, further preferably 5 or less, and particularly preferably 2 or 1 amino acid.

Furthermore, a gene comprising DNA capable of hybridizing under stringent conditions to DNA comprising a nucleotide sequence complementary to DNA comprising the nucleotide sequence of the above gene is also included herein as an example of the gene of the present invention, as long as such a gene encodes a protein having plant environmental stress responsive transcriptional activity. “Stringent conditions” used herein refers to conditions consisting of a formamide concentration ranging from 30% to 50%, 37° C. to 50° C., and 6×SSC and preferably conditions consisting of formamide concentration of 50%, 42° C., and 6×SSC.

Mutation can be introduced into the gene of the present invention by a known technique or a method according thereto, such as the Kunkel method or the Gapped duplex method using a mutagenesis kit (e.g., Mutant-K (produced by TAKARA) or Mutant-G (produced by TAKARA)) or a LA PCR in vitro Mutagenesis series kit (TAKARA), to which site-directed mutagenesis has been applied.

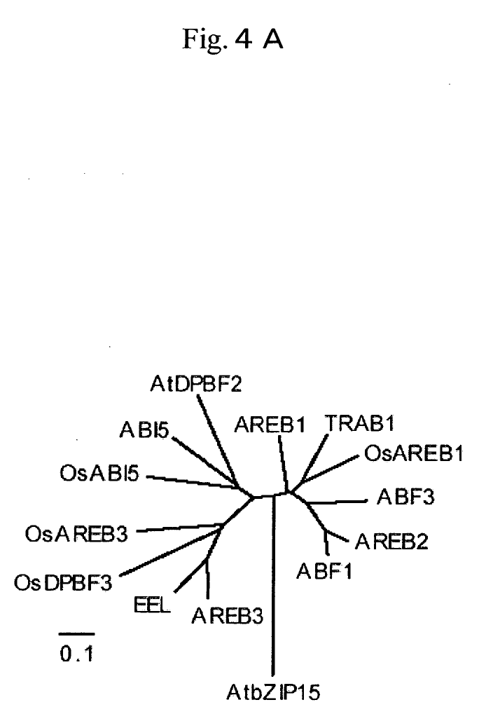

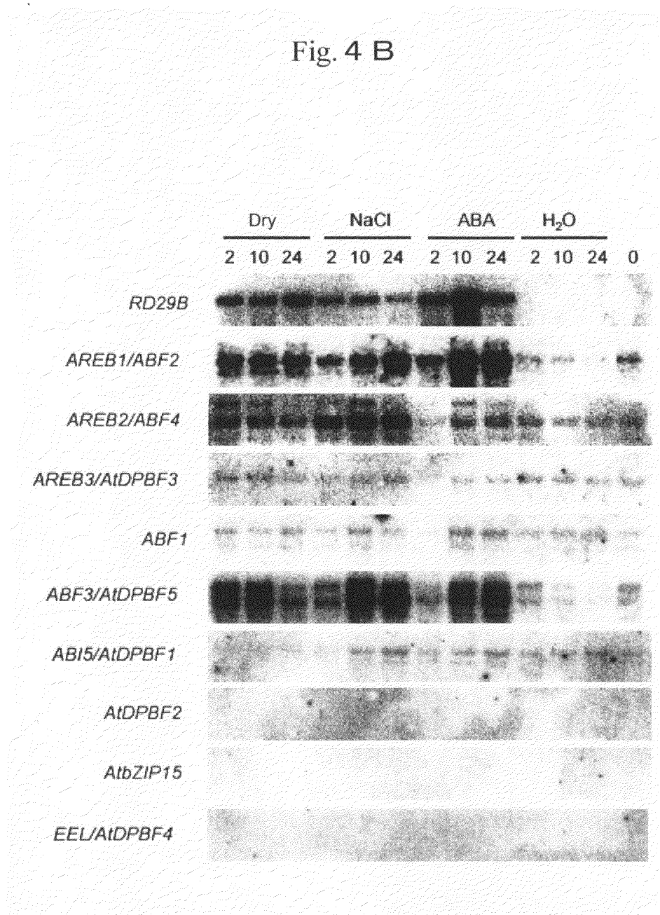

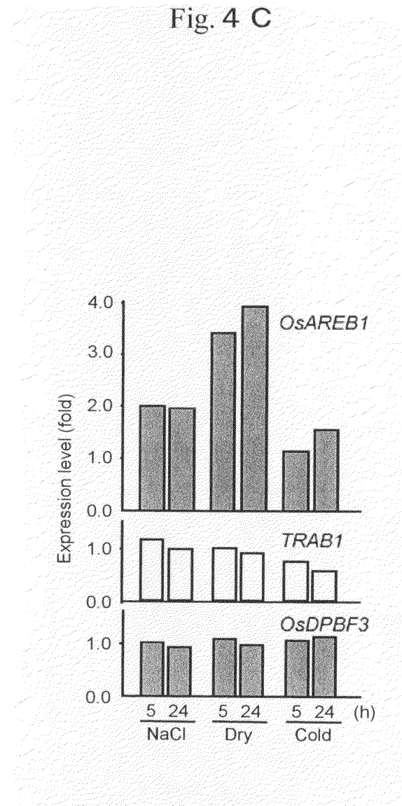

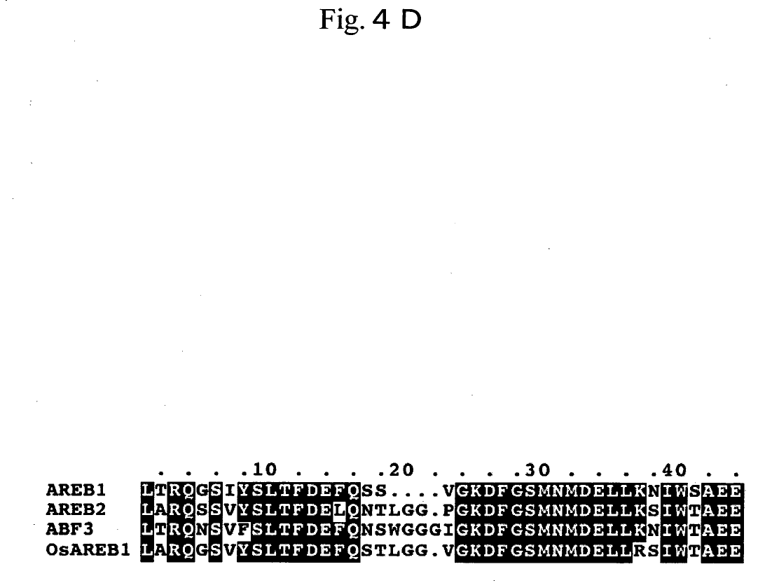

Plant species from which the gene of the present invention is derived is not particularly limited. Examples of the gene of the present invention include an orthologous gene of AREB1 of a dicotyledon such as Arabidopsis thaliana lacking the activation control region and containing the N-terminal transcriptional activation domain and a DNA binding domain and an orthologous gene of AREB1 of a monocotyledon such as rice lacking the activation control region and containing the N-terminal transcriptional activation domain and a DNA binding domain. When the gene of the present invention is derived from plant species other than Arabidopsis thaliana, a region to be deleted corresponds to a region to be deleted from the AREB1 gene of Arabidopsis thaliana. Such region can be determined through alignment of Arabidopsis thaliana-derived AREB1 gene with the orthologous gene of AREB1 derived from another plant species. For example, FIG. 4D shows the alignment of a partial amino acid sequence of the transcriptional activity region of Arabidopsis thaliana AREB1 protein with that of rice AREB1 (OsAREB1) protein. The rice-derived OsAREB1 nucleotide sequence is shown in SEQ ID NO: 9 and the OsAREB1 amino acid sequence is shown in SEQ ID NO: 10. In the OsAREB1 protein, P region is a site ranging from methionine 1 to glutamic acid 63, Q region is a site ranging from serine 64 to serine 126, R region is a site ranging from threonine 127 to leucine 192, S region is a site ranging from phenylalanine 193 to serine 236, T region is a site ranging from asparagine 237 to glutamic acid 276, and U region is a site ranging from arginine 277 to tryptophan 357. Specifically, an example of the gene of the present invention is a gene encoding a protein that lacks an amino acid sequence comprising sequential 100, 150, 200, 250, or 300 amino acids starting from amino acids 50 to 100, 60 to 70, or 100 to 130 among amino acids 64 to 357 of the amino acid sequence represented by SEQ ID NO: 10 of rice-derived OsAREB1 protein. Examples of such protein include: a protein comprising an amino acid sequence derived from the amino acid sequence represented by SEQ ID NO: 2 by deletion of an amino acid sequence ranging from any one of amino acids 60 to 70 to any one of amino acids 270 to 280; a protein comprising an amino acid sequence derived from the same by deletion of an amino acid sequence ranging from any one of amino acids 60 to 100 to any one of amino acids 260 to 290; and a protein comprising an amino acid sequence derived from the same by deletion of an amino acid sequence ranging from any one of amino acids 100 to 130 to any one of amino acids 260 to 290. A preferable example of the same is a protein comprising an amino acid sequence derived from the same by deletion of an amino acid sequence ranging from amino acids 64 to 276.

Furthermore, an example of the gene of the present invention is a gene comprising a nucleotide sequence derived from the nucleotide sequence represented by SEQ ID NO: 9 of rice OsAREB1 gene by deletion of sequential 300, 450, 600, 750, or 900 nucleotides (nucleotide sequence) that start from nucleotides at positions 360 to 520, nucleotides at positions 390 to 430, or nucleotides at positions 510 to 610. Examples of such gene include: a gene comprising a nucleotide sequence derived from the nucleotide sequence represented by SEQ ID NO: 9 by deletion of a nucleotide sequence ranging from any one of nucleotides at positions 390 to 420 to any one of nucleotides at positions 1030 to 1060; a gene comprising a nucleotide sequence derived from the same by deletion of a nucleotide sequence ranging from any one of nucleotides at positions 390 to 510 to any one of nucleotides at positions 1000 to 1090; and a gene comprising a nucleotide sequence derived from the same by deletion of a nucleotide sequence ranging from any one of nucleotides at positions 510 to 600 to any one of nucleotides at positions 1000 to 1090. A preferable example of the same is a gene comprising a nucleotide sequence derived from the same by deletion of a nucleotide sequence ranging from nucleotides at positions 406 to 1045.

The transcriptional activation ability of the protein of the present invention can be analyzed by a transactivation experimental method using an Arabidopsis thaliana protoplast system. For example, AREB1 cDNA is ligated to a pBI221plasmid (produced by Clonetech) containing a CaMV35S promoter, so as to construct an effector plasmid. Meanwhile, a DNA fragment containing ABRE is ligated further upstream of TATA promoter located upstream of a β-glucuronidase (GUS) gene, so as to construct a reporter plasmid. Subsequently, the two types of plasmid are introduced into an Arabidopsis thaliana protoplast and then GUS activity is determined. If an enhancement in GUS activity is observed via simultaneous expression of an AREB1 protein, it can be confirmed that the AREB1 protein expressed within the protoplast activates transcription via the ABRE sequence.

In the present invention, protoplast preparation and introduction of a plasmid DNA into the protoplasts can be performed by the method of Abel et al [Abel, S.: Plant J. 5: 421-427 (1994)]. β-glucuronidase activity can be determined by the method of Jefferson et al [Jefferson, R. A.: EMBO J. 83: 8447-8451 (1986)] and luciferase activity can be determined using a PicaGeneluciferase assay kit (produced by Toyo-Ink).

Wild-type AREB1 does not induce the transcriptional activity of a gene downstream of ABRE, unless otherwise in the presence of abscisic acid. The active AREB1 of the present invention can induce the transcriptional activity of a gene downstream of ABRE even in the absence of abscisic acid.

### 2. Production of Transgenic Plant Via Introduction of the Gene of the Present Invention

A transgenic plant having resistance against environmental stresses, such as drought tolerance, salt tolerance, or low-temperature tolerance can be produced by introducing DNA encoding the protein of the present invention into a host plant with the use of genetic engineering techniques. Examples of a method for introducing the gene of the present invention into a host plant include an indirect introduction method such as an Agrobacterium infection method and a direct introduction method such as a particle gun method, a polyethylene glycol method, a liposome method, and a microinjection method. When the Agrobacterium infection method is used, a plant in which the gene of the present invention has been introduced can be produced as follows.

(1) Construction of Recombinant Vectors for Introduction into Plants and Transformation of Agrobacterium

A recombinant vector for introduction into plants can be obtained by cleaving DNA containing the gene of the present invention with an appropriate restriction enzyme, ligating an appropriate linker if necessary, and then inserting the resultant into a cloning vector for plant cells. As vectors for cloning, binary vector system plasmids such as pBE2113Not, pBI2113Not, pBI2113, pBI101, pBI121, pGA482, pGAH, and pBIG and intermediate vector system plasmids such as pLGV23Neo, pNCAT, and pMON200 can be used.

When such a binary vector system plasmid is used, a target gene is inserted between boundary sequences (LB, RB) of the above binary vector and then the recombinant vector is amplified in Escherichia coli. The thus amplified recombinant vector is then introduced into Agrobacterium tumefaciens C58, LBA4404, EHA101, C58C1Rif^{R}, EHA105, or the like by a freezing and thawing method, an electroporation method, or the like, and then the Agrobacterium is used for plant transduction.

Agrobacterium for plant infection containing the gene of the present invention can also be prepared in the present invention by a three-member conjugation method [Nucleic Acids Research, 12:8711 (1984)] in addition to the above methods. Specifically, Escherichia coli carrying a plasmid that contains a target gene, Escherichia coli carrying a helper plasmid (e.g., pRK2013), and Agrobacterium are subjected to mixed culture and then subjected to culture on medium containing rifampicin and kanamycin, so that conjugate Agrobacterium for plant infection can be obtained.

Expression of a foreign gene or the like in a plant body requires the arrangement of a promoter, a terminator, and the like for plants before and behind the structural gene. Examples of a promoter that can be used in the present invention include promoters for a cauliflower mosaic virus (CaMV)-derived 35S transcript [Jefferson, R. A. et al.: The EMBO J. 6: 3901-3907 (1987)], maize ubiquitin [Christensen, A. H. et al.: Plant Mol. Biol. 18:675-689 (1992)], nopaline synthase (NOS) gene, octopine (OCT) synthase gene, and the like. Examples of such a terminator sequence include a cauliflower mosaic virus-derived terminator and a nopaline synthase gene-derived terminator. However, such examples are not limited thereto, as long as they are promoters or terminators that are known to be able to function within plant bodies.

If a promoter to be used herein is a promoter (e.g., CaMV35S promoter) that is responsible for the constitutive expression of a target gene, so as to cause delayed growth of or dwarfing in gene-introduced plants, a promoter (e.g., rd29A gene promoter) that cause the transient expression of the target gene can be used. Furthermore, an intron sequence (e.g., a maize alcohol dehydrogenase (Adh1) intron [Genes & Development 1: 1183-1200 (1987)]) capable of enhancing gene expression can be introduced between a promoter sequence and the gene of the present invention, according to need.

Moreover, it is preferable to use an effective selection marker gene with the gene of the present invention for efficient selection of target transformed cells. As selection markers to be used in such case, one or more genes selected from among a kanamycin resistance gene (NPTII), a hygromycin phosphotransferase (htp) gene that imparts resistance against antibiotic hygromycin to plants, a phosphinothricinacetyltransferase (bar) gene that imparts resistance against bialaphos to plants, and the like can be used. The gene of the present invention and a selection marker gene can be incorporated together into a single vector or two types of recombinant DNA incorporated into different vectors may also be used.

(2) Introduction of the Gene of the Present Invention into Hosts

Transformant hosts to be used in the present invention are not particularly limited, but are preferably plants. Such plants may be any of cultured plant cells, entire plant bodies of cultivated plants, plant organs (e.g., leaves, petals, stems, roots, root stems, and seeds), or plant tissues (e.g., epidermis, phloem, parenchyma, xylem, and vascular bundle). Plant species are not limited and dicotyledons and monocotyledons such as rice, maize, and wheat can be used. When a plant to be transformed is a dicotyledon, introduction of the gene of the present invention derived from a dicotyledon (e.g., Arabidopsis thaliana) is preferred. When a plant to be transformed is a monocotyledon, introduction of the gene of the present invention derived from a monocotyledon (e.g., rice) is preferred. When cultured plant cells, plant bodies, plant organs, or plant tissues are used as hosts, such host plants can be transformed with DNA encoding the protein of the present invention by introducing a vector into collected plant sections by an Agrobacterium infection method, a particle gun method, a polyethylene glycol method, or the like. Alternatively, such DNA is introduced into protoplasts by an electroporation method, so that transformed plants can also be produced.

For example, when a gene is introduced into Arabidopsis thaliana by the Agrobacterium infection method, a step of infecting a plant with Agrobacterium carrying a plasmid that contains the target gene is essential. This step can be performed by a vacuum infiltration method [CR Acad. Sci. Paris, Life Science, 316: 1194 (1993)]. Specifically, Arabidopsis thaliana is grown using soil prepared by mixing vermiculite with perlite in an equivalent amount. The thus grown maize is directly dipped in a culture solution of Agrobacterium containing the plasmid that contains the gene of the present invention. The plants are then put into a desiccator and then subjected to suction using a vacuum pump until the pressure reaches 65 mmHg to 70 mmHg. The plants are then allowed to stand at room temperature for 5 to 10 minutes. The pots are transferred onto trays and then covered with wrap to keep the humidity. The wrap is removed on the next day. The plants are allowed to grow intact and then seeds are harvested.

Subsequently, seeds are seeded on MS agar medium supplemented with an appropriate antibiotic for selection of plant bodies carrying the target genes. Arabidopsis thaliana plants grown on the medium are transferred into pots and then grown. Thus, the seeds of transgenic plants in which the gene of the present invention has been introduced can be obtained. In general, a transgene is similarly introduced into the genome of a host plant. A phenomenon referred to as “position effect” is observed such that transgene expression differs because of different positions into which genes are introduced. Through verification by a Northern method using a DNA fragment of a transgene as a probe, transformants in which a transgene is more strongly expressed can be selected.

Whether or not a target gene is incorporated in transgenic plants in which the gene of the present invention has been introduced and the plants of the next generation of the transgenic plants can be confirmed by extracting DNA from these cells and tissues according to a standard technique and then detecting the introduced gene using the known PCR method or Southern analysis.

### (3) Analysis of Expression Levels and Expression Sites of the Gene of the Present Invention in Plant Tissues

The expression levels and expression sites of the gene of the present invention in transgenic plants in which the gene has been introduced can be analyzed by extracting RNA from these cells and tissues according to a standard technique and then detecting mRNA of the introduced gene using the known RT-PCR method or Northern analysis. Furthermore, the expression levels and expression sites can also be analyzed by directly analyzing the gene products of the present invention by Western analysis or the like using an antibody against the gene product.

(4) Changes in the mRNA Levels of Various Genes within Transgenic Plant Bodies in which the Gene of the Present Invention has been Introduced

Within a transgenic plant body in which the gene of the present invention has been introduced, the gene whose expression level is thought to be varied by the action of the transcription factor of the present invention can be identified by Northern hybridization.

For example, plants grown by hydroponic culture or the like are exposed to environmental stresses for a predetermined period (e.g., 1 to 2 weeks). Examples of such environmental stresses include drought, salt, and low temperature. For example, drought stress (load) can be applied by removing plant bodies from hydroponic culture and then drying them on filter paper for 10 minutes to 24 hours. Salt stress (load) can be applied by replacing the culture solution with a 50 mM to 500 mM NaCl solution, for example, and then keeping the plant bodies in the solution for 10 minutes to 24 hours. Furthermore, low temperature stress (load) can be applied by, for example, in the case of Arabidopsis thaliana, keeping Arabidopsis thaliana at −15° C. to 5° C. for 10 minutes to 24 hours. Temperatures for low-temperature stress (load) can be adequately determined depending on plant species, plant growth stages, and the like.

Total RNAs are prepared from control plants that have not been exposed to stress and plants that have been exposed to environmental stresses and then subjected to electrophoresis. Northern hybridization is then performed using probes for genes the expression of which is to be examined, so that the expression patterns thereof can be analyzed.

### (5) Evaluation of Tolerance of Transgenic Plants to Environmental Stresses

The tolerance against environmental stresses of transgenic plants in which the gene of the present invention has been introduced can be evaluated by, for example, planting transgenic plants in plant pots filled with soil containing vermiculite, perlite, or the like and then examining the survival of the plants when they are exposed to various environmental stresses. Examples of environmental stresses include drought, salt, and low temperature. For example, tolerance against drought stress can be evaluated by not providing water for 2 to 4 weeks and examining the survival of the plants. Moreover, salt stress can be evaluated by, for example, exposing the plants to 100 mM to 600 mM NaCl for 1 hour to 7 days, further growing the plants for 1 to 3 weeks at 20° C. to 35° C., and then examining the survival rate. Furthermore, low-temperature stress can be evaluated by, for example, in the case of Arabidopsis thaliana, exposing the plants to a temperature ranging from −15° C. to 5° C. for 30 minutes to 10 days, growing the plants for 2 days to 3 weeks at 20° C. to 35° C., and then examining the survival rate.

### 3. Measurement of Plant Stress Level

The transcription of the gene of the present invention is activated by drought stress, salt stress, low-temperature stress, or the like, so that the levels of environmental stresses due to drought, salt, and/or low temperature to which plants are exposed can be examined through examination of the transcriptional level of the gene of the present invention.

The transcriptional level of the gene of the present invention can be examined by RNA gel-blot analysis, quantitative PCR, or the like. A probe to be used for RNA gel-blot analysis can be prepared using a known method based on 100-bp to 1000-bp region containing the gene of the present invention and/or a specific sequence adjacent to the gene of the present invention. Primers to be used for quantitative PCR can be prepared by a known method based on a sequence within the coding region of the gene of the present invention or a sequence of a region adjacent thereto.

The present invention will be described in more detail below with reference to examples. However, the present invention is not limited to embodiments described in the examples.

### (1) Methods and Materials

Materials and methods employed in the examples are as described below.

### Plants and Growth Conditions

Plants (Arabidopsis thaliana ecotype Columbia) were grown on GM agar medium for 2 to 3 weeks under light conditions of 16-hour light periods/8-hour dark periods (40±10 μmol photons/m²/s) according to the method of Osakabe et al (2005, Plant Cell 17, 1105-1119) (2005, Plant Cell 17, 1105-1119). 1% or 3% sucrose was added to GM agar medium and a necessary amount of ABA was further added depending on experimentation. Arabidopsis thaliana T87 culture cells were maintained and controlled according to the method of Satoh et al (2004, Plant Cell Physiol. 45, 309-317). A variant line (SALK_{—}002984; Col-0 ecotype) of Arabidopsis thaliana AREB1 into which T-DNA had been inserted was obtained from Arabidopsis Biological Resource Center (Columbus, Okla., U.S.A.). Furthermore, the information concerning the variant into which T-DNA had been inserted was obtained from the web site (http://signal.salk.edu) of Salk Institute Genomic Analysis Laboratory (California, U.S.A.). Furthermore, the site of the AREB1 gene into which T-DNA had been inserted was confirmed by PCR using a T-DNA left border primer 5′-GCGTGGACCGCTTGCTGCAACT-3′ (SEQ ID NO: 3) and an AREB1-specific primer 5′-TCAAGCTCCACGGTGTAAGCC-3′ (SEQ ID NO: 4). Moreover, analysis using the RT-PCR method was performed according to the method of Ito and Shinozaki (2002, Plant Cell Physiol. 43, 1285-1292), so as to confirm that the AREB1 gene had not been expressed in the areb1 variant line into which T-DNA had been inserted.

### RNA Gel Blot Analysis

Extraction of total RNA from plant bodies and RNA gel blot analysis using the total RNA were performed according to the method of Satoh et al (2004, Plant Cell Physiol. 45, 309-317) using a Shakemaster crusher (Bio Medical Science, Tokyo, Japan). Furthermore, probes for RNA gel blot analysis were prepared according to the method of Maruyama et al (2004, Plant J. 38, 982-993).

Analysis of Transient Expression Using Arabidopsis thaliana Protoplasts

Analysis of transient expression was performed using protoplasts derived from Arabidopsis thaliana T87 cells according to a slightly modified version of the method of Fujita et al (2004, Plant J. 39, 863-876) and the method of Satoh et al (2004, Plant Cell Physiol. 45, 309-317). Protoplast isolation and gene introduction were all performed at room temperature (25° C. to 28° C.). Protoplasts in which the gene had been introduced were allowed to stand for 16 to 20 hours at 22° C. in the dark. An enzyme solution [0.4 M mannitol, 1.5% (w/v) cellulase “Onozuka” R-10 (Yakult, Tokyo, Japan), 0.3% (w/v) macerozyme R-10 (Yakult), 0.1% (w/v) bovine serum albumin, 10 mM CaCl₂, 20 mM KCl, and 20 mM MES, pH 5.7] was prepared according to the method of Sheen (2002) (http://genetics.mgh.harvard.edu/sheenweb/).

A DNA fragment containing a portion or the whole of AREB1 cDNA was amplified by PCR and then the product was cloned into the Not I site of an expression vector pBI35SΩ (Abe et al., 1997, Plant Cell 9, 1859-1868). Thus, a plasmid having an AREB1 bZIP DNA binding domain was prepared. The plasmid was then used for an experiment for the analysis of transient expression. To remove a 640-base-pair fragment that was partially cleaved with EcoT14I from the pBI35SΩ-AREB1 plasmid, self-binding ligase reaction was performed after partial cleavage with EcoT14I. The thus prepared pBI35SΩ-AREB1ΔQT was a plasmid containing an internal deletion (amino acid residues 65-277) ranging from the Q to the T region. Two DNA fragments containing portions of AREB1 cDNA were prepared by PCR using the following two sets of primers: a forward primer A, 5′-GGGGCGGCCGCATGACACAAGCCATGGCTAGTG-3′ (SEQ ID NO: 5); a reverse primer A, 5′-GCAGAAGCACCTTGACTTCCCCCTACTCCAC-3′ (SEQ ID NO: 6); a forward primer B, 5′-GTAGGGGGAAGTCAAGGTGCTTCTGCTGC-3′ (SEQ ID NO: 7); and a reverse primer B, 5′-GGGGAGCTCTCACCAAGGTCCCGACTCTG-3′ (SEQ ID NO: 8). The thus prepared fragments A and B were mixed within a tube for PCR, denatured at 94° C. for 10 minutes, and then subjected to annealing and polymerase reaction at 72° C. for 3 minutes. The DNA fragment that had been amplified using forward primer A and reverse primer B was introduced into pBI35SΩ. The thus prepared pBI35SΩ-AREB1ΔP/RT was a plasmid containing two internal deletion portions (amino acid residues 1-60 and 117-277).

An expression plasmid (p35S-562) containing a GAL4 DNA binding domain bound to a GAL4 activation domain and a GAL4-GUS reporter plasmid (pGUS-558) used herein had been provided by Dr. Tsukaho Hattori (Nagoya university, Japan). The expression plasmid containing the GAL4 binding domain used in the experiment for analysis of transient expression was prepared by cloning a PCR fragment containing a portion of AREB1 cDNA into the BamH I/Sac I site of the expression vector p35S-562. A 900-base-pair Hind III/BamH I fragment obtained from pBI-35SLUC (Urao et al., 1996, Plant J. 10, 1145-1148) was inserted into the Hind III/BamH I site of a plant expression vector pBI221-(−46/Ω)LUC provided by Dr. Takeshi Urao (Japan International Research Center for Agricultural Sciences, Japan). The thus prepared pBI35SΩ-LUC was used as an internal standard for an experiment concerning transient transcriptional activation.

### Constructs for Transformed Plants

To prepare a pBE2113Not-AREB1 plasmid, the entire coding region of AREB1 was amplified by PCR using a primer with a Not I linker and then the resultant was introduced into pBE2113Not (Liu et al., 1998, Plant Cell 10, 1391-1406) in the sense orientation. pBE2113Not-AREB1ΔQT was prepared by amplifying the coding region of AREB1ΔQT by PCR using primers with Xba I and BamH I linkers and then introducing the resultant into the Xba I/BamH I site of pBE2113Not in the sense orientation.

To produce transformed plants with 35S-AREB1 and 35S-AREB1ΔQT, transformation vectors, pBE2113Not-AREB1 and pBE2113Not-AREB1ΔQT, were introduced into Arabidopsis thaliana (Columbia) by a suction-infiltration method (Osakabe et al., 2005, Plant Cell 17, 1105-1119) using the Agrobacterium tumefaciens C58 strain.

### Drought Tolerance Test

The drought tolerance test was conducted using a slightly modified version of the method of Sakamoto et al (2004, Plant Physiol. 136, 2734-2746). Plants were grown under light conditions of 16 hour-light periods/8-hour dark periods (50±10 μmol photons/m²/s) and conditions of 22° C.±1° C. and relative humidity of 35±5%. These plants were exposed to drought stress by halting the supply of water to them for 12 days.

For the plant survival test under acute drought conditions, plants that had been seeded on GM agar medium and then grown for 2 to 3 weeks were used. These plants were carefully removed from agar medium and then transferred onto Petri dishes (without covers). The plants were then dried for a predetermined time. After completion of drying, water was supplied. The survival test under acute drought conditions was conducted under light conditions of 9±1 μmol photons/s/m² at 25° C.±2° C. (relative humidity of 20%±10%). After water was supplied again, Petri dishes containing the plants were transferred into a plant culture room and then the plants were allowed to stand at 22° C.±2° C. under continuous light irradiation (50±5 μmol photons/s/m²) for 1 to 3 days. Survival or death was determined based on plant color. Each plant in which 50% or more of the tissue was green, was determined to be alive. Plants of almost the same size were used to minimize the effects resulting from the size of plant bodies. All experiments were repeated at least 5 times. At least 3 lines and 40 or more plant bodies were used for each comparative experiment.

### Microarray Analysis

35S-AREB1ΔQT and vector control plant bodies that had been grown on GM agar medium for 2 weeks were directly collected or collected after 7 hours of treatment with ABA. The plant bodies were then analyzed using an Agilent Arabidopsis 2 Oligo Microarray (Agilent Technologies, Palo Alto, Calif., U.S.A.). For biological repetition, 8 individual plant bodies were used as a sample for RNA extraction. Individual two lines of transformants were used for each experiment. Total RNA was extracted with a Trizol reagent (Invitrogen) and then used for preparation of Cy5- and Cy3-labeled cDNA probes. All microarray experiments including data analysis were conducted according to the protocols provided with the products (http://www chem.agilent.com/scripts/generic asp?1page=11617&indcol=Nandprodcol=Y). For evaluation of reproducibility of microarray analysis, a confirmation experiment was conducted for each experiment. Specifically, it was confirmed herein whether the same result could be obtained even when a dye was replaced by another dye (Cy5 and Cy3). Our experimental findings include a case in which reproducibility of the RNA gel blot analysis could not always be confirmed for a control plant with a signal intensity of Cy5 or Cy3 ranging from 500 to 1000 or less. Therefore, under our experimental conditions, the genes of control plants showing signal intensities of 1000 or less in cases involving Cy5 and Cy3 were not used for the analysis. Furthermore, we conducted analysis for those with P values of 0.001 or less. Moreover, it could be confirmed based on our past research data that changes in gene expression could be significantly confirmed with good reproducibility by RNA gel blot analysis and quantitative real-time RT-PCR analysis in the case of the genes found to exhibit changes in gene expression 3 or more times greater in a microarray experiment (e.g., Rabbani et al., 2003, Plant Physiol. 133, 1755-1767; Fujita et al., 2004, Plant J. 39, 863-876; and Maruyama et al., 2004, Plant J. 3, 982-993). Each spot on the array was specified and quantified using feature extraction and image analysis software (version A.6.1.1; Agilent Technologies), followed by standardization by the Lowess method. Genespring 6.1 software (Silicon Genetics, San Carlos, Calif., U.S.A.) was used for gene clustering analysis. At4g25580 existing in the Arabidopsis thaliana genome contains a nucleotide sequence that is very similar to that of RD29B. It was confirmed that RD29B gene expression alone was increased in 35S-AREB1ΔQT plant bodies by quantitative real-time RT-PCR analysis and RNA gel blot analysis using a sequence specific to RD29B.

### Analysis Based on Water Relations in Plant Physiology

The amounts of water missing from the plant bodies and relative water contents of the same were measured by a slightly modified version of the method of Yoshida et al (2002, Plant Cell Physiol. 43, 1473-1483) and Ma et al (2004, Plant J. 40, 845-859). Above-the-ground portions of plant bodies (that had been grown for 4 weeks in pots filled with soil) were cut off and then the fresh weights thereof were measured over time. The above-the-ground portions cut off from the plant bodies were dried for a predetermined time and then subjected to dry-heat treatment at 180° C. for 3.5 hours. The dry weights thereof were measured. The relative water content was calculated using the formula of [(FWᵢ−DW)/(FW₀−DW)]×100 to minimize the effect of variation in values due to plant size or dry weight. The relative water content was expressed with a percentage of the initial water content of the above-the-ground portion. Fwᵢ and FW₀ indicate the fresh weight after a specific time and the initial fresh weight, respectively. DW indicates the dry weight. These tests were conducted on a bench in an experimental room (24° C.±1° C., relative humidity of 65%±5%, and light conditions of 9±1 μmol photons/s/m²).

### Analysis of Phylogenetic Tree

An alignment was prepared from three N-terminal conserved regions (C1, C2, and C3: FIG. 1A) and the sequence of a bZIP region using a ClustaIX program (version 1.83). At this time, variables were as determined below: gap open penalty=5.00 and gap extension penalty=0.05 (Supplemental FIG. 1). In addition, the alignment was finally subjected to manual fine adjustment. A phylogenetic tree was produced according to the method of Fujita et al (2004, Plant J. 39, 863-876); it involved a neighbor-joining method using MEGA software (version 3). Monophyletic group reliability was calculated by bootstrap analysis (repeated 1000 times).

(2) Examination of Transactivation Activity of AREB1 N-Terminal Conserved Region Using Protoplasts Derived from Arabidopsis thaliana T87 Culture Cells AREB1

In previous studies, the present inventors have demonstrated that the AREB1 protein activates the transcription of the RD29B promoter-GUS fusion gene (RD29B-GUS) using Arabidopsis thaliana protoplasts (Uno et al., 2000, Proc. Natl. Acad. Sci. U.S.A. 97, 11632-11637). This time, in order to identify the AREB1 transcriptional activation domain, the present inventors prepared a plasmid to cause constant expression of deficient AREB1 having a deletion at the N-terminus with the use of a cauliflower mosaic virus 35S promoter (FIG. 1). The expression plasmid was introduced together with a reporter plasmid RD29B-GUS into protoplasts prepared from Arabidopsis thaliana T87 culture cells. The reporter plasmid has a sequence in which five sets of 77 base pairs including two ABREs within the RD29B promoter are serially aligned and connected to the GUS reporter gene (FIG. 1A).

FIG. 1A shows the constructs of an effector plasmid and a reporter plasmid. A plasmid used as the reporter plasmid was prepared by repeatedly (5 times) arranging a 77-bp ABRE sequence; that is, the RD29B promoter upstream of the GUS gene. The promoter was ligated upstream of a −51RD29B minimal TATA promoter-GUS construct. Nos-T is a nopaline synthase terminator. A plasmid used as the effector plasmid was prepared by inserting the full-length of AREB1 or a portion of AREB1 containing the bZIP DNA binding domain downstream of the CaMV35S-TMV Ω sequence. These two plasmids were simultaneously introduced into protoplasts of Arabidopsis thaliana T87 cells. The AREB1 protein, synthesized in the protoplasts binds to the ABRE sequence of the reporter plasmid so as to induce transcription of the GUS gene located downstream. Such transcription-inducing activity of the AREB1 protein can be obtained by measuring GUS protein activity.

When a short region (P region) of 60 amino acid residues located on the N-terminus of AREB1 was deleted, the transcriptional activity of the reporter gene in protoplasts was found to have been significantly decreased, regardless of whether or not 100 μM ABA had been added, suggesting the presence of a positive control domain within the region (FIG. 1B).

FIG. 1B shows the results of analyzing the transactivation domain of AREB1 from which the N-terminus has been deleted. Protoplasts were co-transfected with an RD29B-GUS reporter construct and an effector construct. The vector used herein was pBI-35SQ. Relative activity is represented by the ratio of the expression level of the AREB1 protein to the expression level of the pBI-221-35SΩ control. Numerical figures in FIG. 1B denote amino acid numbers. P, Q, R, S, T, and U denote partial regions of AREB1 cDNA, and black portions of P, Q, R, and U regions denote conserved regions. In the presence of 50 μM abscisic acid (ABA), a significant enhancement in GUS activity was observed in the case of the full-length AREB1 protein, demonstrating that AREB1 functions to activate transcription induction for AREB1. Meanwhile, when the N-terminal region and a region of amino acids 1-60 had been deleted from the AREB1 protein, GUS activity was found to decrease to the background level even in the presence of ABA. The presence of the transcriptional activation domain of the AREB1 protein within the region was suggested.

The present inventors further examined whether or not a variant protein having an AREB1 P region and a binding domain activated the transcription of the RD29B-GUS reporter gene in the absence of external ABA. As expression plasmids, AREB1Δ QT and AREB1AP/RT, each having an AREB1 bZIP DNA binding domain and the P or Q region, were prepared (FIG. 1C). FIG. 1C shows the results of transactivation of AREB1ΔQT and AREB1ΔP/RT. In the case of an AREB1 variant (AREB1ΔQT) from which the region of amino acids 65-277 had been deleted, transcriptional activity was significantly induced, even in the absence of ABA. Because of the simultaneous introduction of AREB1ΔQT and RD29B-GUS, the GUS reporter gene was significantly activated even in the absence of ABA. However, in the case of AREB1ΔP/RT, the reporter gene was not activated, regardless of whether ABA was present or absent. These results suggest that the P region on the N-terminus of AREB1 contains the transcriptional activation domain and that AREB1ΔQT is constantly in an activated form in protoplasts.

Furthermore, in the cases of many AREB1 variants in which AREB1ΔP/RT or other N-termini had been deleted, transcriptional activation ability was significantly decreased compared with the vector control in the presence of ABA. Such decreases in transcriptional activation ability observed in these variants may be caused as follows. AREB1 variants from which AREB1ΔP/RT or N-termini had been deleted bound preferentially to the ABRE motif within reporter plasmids, so as to hinder the binding of ABA-induced endogenous transcription factor (originally existing in plant cells) to the ABRE motif (FIG. 1B and FIG. 1C). However, among N-terminus-deficient variants, only in the case of the P-region-deficient variant, almost no suppression of the activation of the reporter gene in the presence of ABA was observed. A possible reason for this is that the sequence of the P-region-deficient variant could specifically exert some sort of effect.

In addition, all transactivation experiments were repeated 3 to 10 times. Typical results of one experiment are shown. Bars in the bar graph denote standard deviations (n=3 to 5).

### (3) Examination of AREB1ΔQT Expression

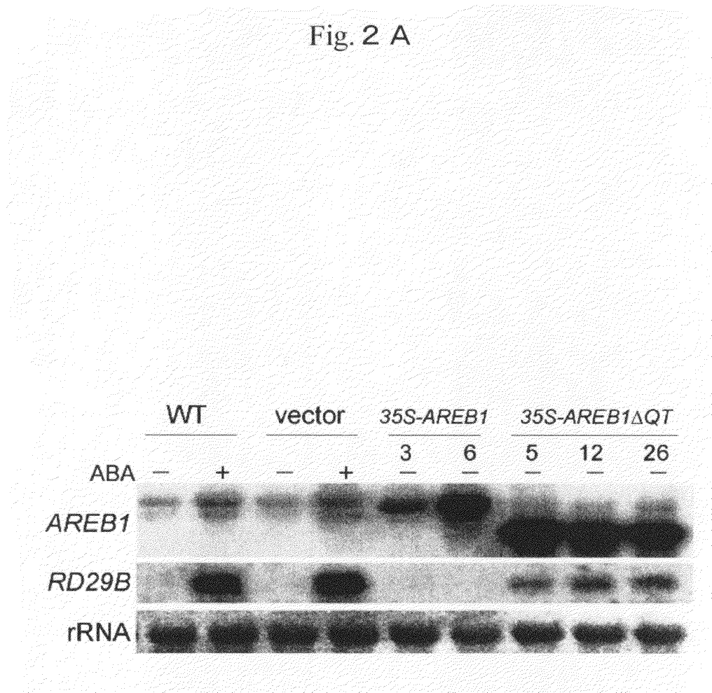

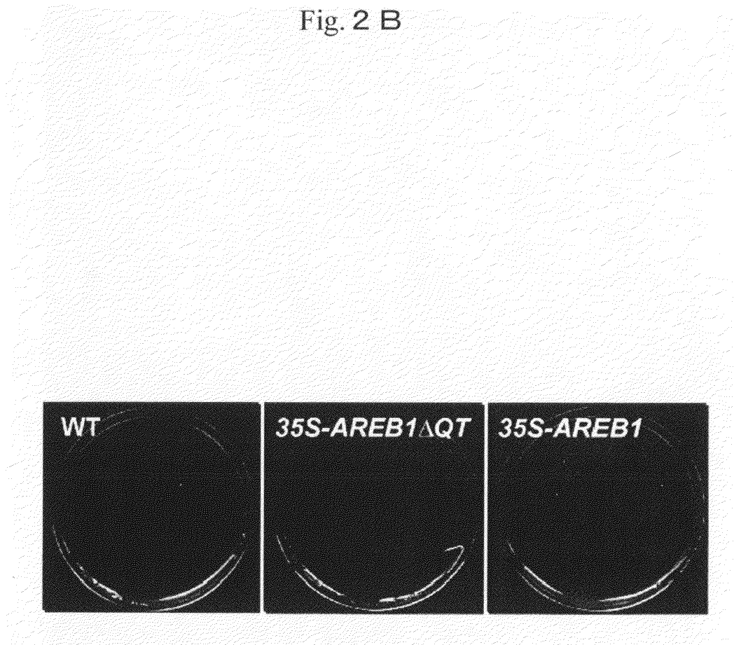

The present inventors have demonstrated that the AREB1 protein binds to two ABRE sequences in the RD29B promoter, so as to activate gene expression (Uno et al., 2000, Proc. Natl. Acad. Sci. U.S.A. 97, 11632-11637). Meanwhile, as described previously, in the absence of external ABA, ABRE1 did not activate the expression of the downstream RD29B gene. Here, it was examined whether or not overexpression of ABRE1ΔQT in Arabidopsis thaliana would activate the transcription of a downstream gene such as RD29B in the absence of external ABA. First, transformed plants were produced by causing expression of ABRE1ΔQT cDNA using a cauliflower mosaic virus 35S promoter. The 33 individual lines of the transformants were subjected to analysis of the expression of the transgene ABRE1ΔQT and the downstream gene RD29B without performing any stress treatment. Under conditions in which no ABA was externally added, the accumulation levels of ABRE1ΔQT and RD29B were found to increase in all the transformant lines examined. For phenotype analysis, eight lines with high expression levels of ABRE1ΔQT were selected. In addition, different lines were used in experimentation because the number of seeds of each line was insufficient for statistical analysis and because the phenotypes of the eight lines were similar. FIG. 2 shows the results of RNA gel-blot analysis of the wild-type and the vector control. Active AREB1 (AREB1ΔQT) that constantly induces transcriptional activity in the absence of ABA (as confirmed in the experiments of FIG. 1) was expressed in Arabidopsis thaliana using a CaMV 35S promoter and a TMV 0 sequence. 2-week-old seedlings were treated for 7 hours or not treated with abscisic acid. FIG. 2A shows the results of Northern analysis. Each lane contains 10 μg of total RNA. The bottom portion of FIG. 2A shows a control stained with ethidium bromide. As a result of Northern analysis, in the cases of the wild-type (WT) and vector control plants (vector), the expression of the downstream gene RD29B having the ABRE sequence in the promoter region could be confirmed only in the presence of 50 μM ABA. Meanwhile, whereas the expression of the RD29B gene was not observed in the absence of ABA in the case of plants (35S-AREB1) caused to express full-length AREB1 cDNA, the expression of the downstream RD29B gene was induced even in the absence of ABA in the case of plants (35S-AREB1ΔQT) caused to express active AREB1. ABRE1ΔQT over-expressed in plants activated, in the absence of external ABA, transcription of RD29B, the downstream gene. These results demonstrated that ABRE1ΔQT is a constantly activated form of ABRE1 of overall plants and that the P region located on the N-terminus of ABRE1 also functions as a transcriptional activation domain in plant bodies in a manner similar to the case of protoplasts.

FIG. 2B shows growth on GMK medium containing 1% sucrose using photographs taken on week 3 after seeding. When full-length AREB1 cDNA was expressed in Arabidopsis thaliana (35S-AREB1), almost no difference was observed in growth compared with the control plants (WT). However, delayed growth was observed to some extent in the case of plants (35S-AREB1ΔQT) caused to express active AREB1. This is a phenomenon observed among DREB1A-expressing plants having improved environmental stress tolerance. At week 3 after seeding, the “Maximum radius of the Rosetta leaves of 35S-ABRE1ΔQT plants accounted for 70% of that of the wild-type plants in average. Throughout all the growth processes, 35S-ABRE1ΔQT plants were slightly smaller than the wild-type plants; however, the 35S-ABRE1 plants showed phenotypes similar to those of the wild-type plants in terms of growth.

(4) Microarray Analysis of Arabidopsis thaliana Expressing Active AREB1

Microarray analysis was performed using Arabidopsis thaliana (35S-AREB1ΔQT) expressing active AREB1. As a microarray, an Agilent Arabidopsis 2 oligo microarray (Agilent Technologies) capable of examining the expression profile of 85% or more of the genes of Arabidopsis thaliana was used. When no treatment had been performed in the absence of ABA, the expression of eight genes of plants expressing active AREB1 was induced at levels 4 or more times higher than those of the same genes in the vector control plants (Table 1). Four out of the eight downstream genes were LEA protein genes involved in water stress tolerance and the remaining four genes were genes involved in signal control. These downstream genes were all ABA- and drought-induced genes. In the promoter regions of these genes, two or more ABRE sequences essential for binding of AREB1 protein were present.

TABLE 1
Table 1. Genes up-regulated in plants overexpressing AREB1ΔQT, identified by microarray analysis.

| | | | | | | Experiment 1 | | | | Experiment 2 | | | | | |
|---|---|---|---|---|---|---|---|---|---|---|---|---|---|---|---|
| | | | | | | (Transgenic line 8) | | | | (Transgenic line 12) | | | | | |
| | | | | | | Ratio | P-value | Ratio | P-value | Ratio | P-value | Ratio | P-value | | No. |
| Inducibility^{a} | | | | Gene | Description^{b} | 1 | 1 | 2 | 2 | 1 | 1 | 2 | 2 | AGI code | ABREs^{c} |
| D | — | A | — | HIS 1-3 | linker histone H1 | 17.6 | 5.6E−32 | 20.9 | 1.5E−32 | 12.1 | 3.2E−30 | 13.3 | 1.1E−30 | At2g18050 | 2 |
| D | S | A | — | AIA1 | AAA family | 12.8 | 9.1E−30 | 8.6 | 7.0E−26 | 13.9 | 1.1E−30 | 16.1 | 1.2E−30 | At1g64110 | 2 |
| | | | | | ATPase | | | | | | | | | | |
| D | S | A | — | AIL1 | LEA class protein^{d} | 9.8 | 1.2E−28 | 8.5 | 4.0E−27 | 28.0 | 2.2E−33 | 16.2 | 1.5E−31 | At3g17520 | 2 |
| D | S | A | — | RD29B | LEA class protein^{d} | 9.8 | 2.0E−27 | 4.6 | 2.1E−18 | 7.3 | 6.5E−25 | 6.0 | 7.2E−21 | At5g52300 | 5 |
| D | S | A | — | GBF3 | transcription factor | 5.2 | 1.7E−22 | 4.0 | 1.2E−18 | 6.9 | 6.7E−26 | 6.2 | 2.6E−24 | At2g46270 | 8 |
| D | S | A | C | RD20 | Ca²+-binding | 6.5 | 1.3E−25 | 7.3 | 1.6E−26 | 4.5 | 1.9E−21 | 4.5 | 3.1E−21 | At2g33380 | 3 |
| | | | | | EF-hand protein | | | | | | | | | | |
| D | S | A | C | RAB18 | LEA class protein^{d} | 5.7 | 3.2E−24 | 4.1 | 5.0E−20 | 6.0 | 8.0E−25 | 5.2 | 4.0E−23 | At5g66400 | 5 |
| D | S | A | C | KIN2 | LEA class protein^{d} | 4.0 | 7.3E−20 | 5.8 | 2.4E−24 | 4.5 | 2.0E−21 | 5.1 | 5.2E−23 | At5g15970 | 6 |

^{a}Data on inducibility were based on microarray analysis (Seki et al., 2002; K. Maruyama, unpublished data). D, drought; S, high salinity; A, ABA; C, cold.
^{b}Description as given by the TIGR database.
^{c}Number of ABRE core sequences in 1000 bp of the sequence upstream of the gene.
^{d}LEA, late embryogenesis abundant.

(5) Drought Stress Tolerance Test for Arabidopsis thaliana Expressing Active AREB1

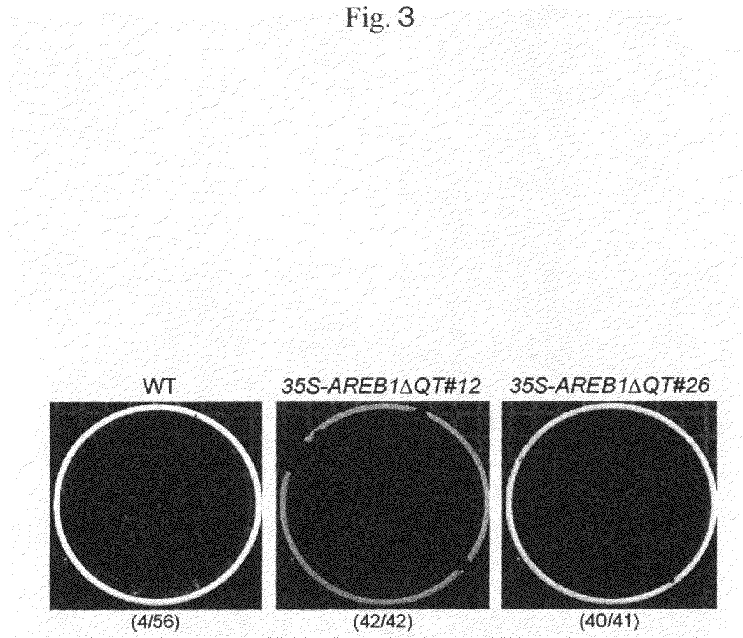

Among stress responsive genes, a gene encoding the LEA protein is thought to be associated with drought tolerance (Ingram and Bartels, 1996, Plant Mol. Biol. 47, 377-403; Thomashow, 1999, Plant Mol. Biol. 50, 571-599). The expression levels of the eight genes were significantly elevated in 35S-ABRE1ΔQT plants under conditions of no stress treatment and four out of the eight genes were LEA proteins. Accordingly, enhanced drought tolerance of 35S-ABRE1ΔQT plants was inferred. To confirm this, the present inventors examined whether ABRE1ΔQT overexpression would affect tolerance against drought stress. Several independent lines were compared but they showed similar phenotypes (data not shown). Then, detailed analysis was performed between lines 12 and 26. The stress tolerance of Arabidopsis thaliana overexpressing active DREB2A was confirmed. A drought tolerance test was conducted by stopping irrigation for 2 weeks for plants at week 4 after seeding. After 2 weeks of the stop of irrigation, to clearly confirm the life or death of plant bodies, irrigation was started again and then photographs were taken 5 days after the re-start of irrigation. Numerical figures under the photographs shown in FIG. 3 indicate the number of plant bodies (denominator) used for the test and the number of plant bodies (numerator) that had survived. In the drought stress experiment, whereas many vector control plants (WT) withered, most plants (35S-AREB1ΔQT) expressing active AREB1 kept their healthy conditions. It was demonstrated that plant's environmental stress tolerance can be improved via expression of active AREB1. As shown in FIG. 3, when water supply was stopped for 12 hours, almost all wild-type plants completely withered. On the other hand, both lines of ABRE1ΔQT plants continued their growth without showing any changes when water supply was re-started. During the period of the drought stress experiment, differences in water content in soil among all pots were within 5% (data not shown). As described above, 35S-ABRE1ΔQT plants exerted high survival capability under acute dehydration conditions and enhanced tolerance to drought stress.

(6) Phylogenetic Tree Analysis of AREB1 Homologous Gene of Arabidopsis thaliana and Rice

FIG. 4A shows the relationship of AREB1 homologous gene of a dicotyledon, Arabidopsis thaliana, with the same of a monocotyledon, rice, using a phylogenetic tree. FIG. 4B shows the gene expression profiles of the AREB1 homologous gene of Arabidopsis thaliana when ABA, drought, salt, and water treatment were performed. Plants that had been grown on GMK medium for 2 weeks after seeding were used in the experiment. A Northern method was performed to show gene expression. Each numerical figure indicates the time for each treatment. FIG. 4C shows the gene expression profiles of the AREB1 homologous gene of rice when drought, salt, and low-temperature treatment were performed. Plants that had been grown by hydroponic culture for 2 weeks after seeding were used in the experiment. A microarray method was performed to show gene expression. Each expression level is represented by the ratio of that of the gene in the plant for comparison to that of the gene in the control plant (the expression level of the control plant is determined to be 1). Each numerical figure shows the time for each treatment. FIG. 4D shows a comparison of Arabidopsis thaliana with rice in terms of the amino acid sequence of the transcriptional activation region in the AREB1 homologous gene.

### INDUSTRIAL APPLICABILITY

Plant drought tolerance, salt tolerance, and low-temperature tolerance can be enhanced by introducing the plant active environmental stress responsive transcription factor AREB1 gene of the present invention lacking the activation control region of the plant transcription factor AREB1 that can respond to environmental stress (e.g., drought stress, salt stress, and low-temperature stress) and containing the N-terminal transcriptional activation domain and a DNA binding domain into a plant and then causing overexpression of the gene.

All publications, patents, and patent applications cited in this description are herein incorporated by reference in their entirety.

### Sequence Listing Free Text

SEQ ID NOS: 3 to 8—primers

1. An active environmental stress responsive transcription factor AREB1 gene, lacking a part or the whole of an activation control region of a plant environmental stress responsive transcription factor AREB1 and containing the N-terminal transcriptional activation domain and a DNA binding domain.

2. An active environmental stress responsive transcription factor AREB1 gene, lacking at least one of the Q, R, S, and T regions of a plant environmental stress responsive transcription factor and containing a P region that contains the N-terminal transcriptional activation domain and an U region that contains a DNA binding domain.

3. The active environmental stress responsive transcription factor AREB1 gene according to claim 1, which is derived from Arabidopsis thaliana.

4. The active environmental stress responsive transcription factor AREB1 gene according to claim 3, encoding a protein that comprises an amino acid sequence derived from the amino acid sequence represented by SEQ ID NO: 2 by deletion of an amino acid sequence ranging from amino acids 65 to 277.

5. An active environmental stress responsive transcription factor AREB1 gene, encoding a protein that comprises an amino acid sequence derived from the amino acid sequence of the active environmental stress responsive transcription factor AREB1 gene according to claim 3 or 4 by deletion, substitution, or addition of one or several amino acids and has plant environmental stress responsive transcriptional activity.

6. The active environmental stress responsive transcription factor AREB1 gene according to claim 3, comprising a nucleotide sequence derived from the nucleotide sequence represented by SEQ ID NO: 1 by deletion of a nucleotide sequence ranging from nucleotides at positions 312 to 950.

7. An active environmental stress responsive transcription factor AREB1 gene, comprising DNA that is capable of hybridizing under stringent conditions to DNA comprising a nucleotide sequence complementary to DNA comprising the nucleotide sequence of the active environmental stress responsive transcription factor AREB1 gene according to claim 6 and encodes a protein having plant environmental stress responsive transcriptional activity.

8. An active environmental stress responsive transcription factor AREB1 gene, lacking an activation control region of an environmental stress responsive transcription factor OsAREB1 derived from rice and containing a transcriptional activation domain and a DNA binding domain.

9. The active environmental stress responsive transcription factor AREB1 gene according to any one of claims 1 to 8, in which the environmental stress is drought stress, salt stress, or low-temperature stress.

10. A plant transformation vector, containing the gene according to any one of claims 1 to 9.

11. A transgenic plant, having enhanced environmental stress tolerance as a result of transformation with the plant transformation vector according to 10.

12. A method for enhancing the environmental stress tolerance of a plant via introduction of the gene according to any one of claims 1 to 9 into the plant.

13. The method for enhancing environmental stress tolerance according to claim 12, in which the environmental stress tolerance is drought tolerance, salt tolerance, and/or low-temperature tolerance.

14. The active environmental stress responsive transcription factor AREB1 gene according to claim 2, which is derived from Arabidopsis thaliana.

15. The active environmental stress responsive transcription factor AREB1 gene according to claim 14, encoding a protein that comprises an amino acid sequence derived from the amino acid sequence represented by SEQ ID NO: 2 by deletion of an amino acid sequence ranging from amino acids 65 to 277.

16. The active environmental stress responsive transcription factor AREB1 gene according to claim 14, comprising a nucleotide sequence derived from the nucleotide sequence represented by SEQ ID NO: 1 by deletion of a nucleotide sequence ranging from nucleotides at positions 312 to 950.


SEQUENCE LISTING (10 sequences)

SEQ ID NO: 1 - DNA, 1662 bp, Arabidopsis thaliana
  CDS (120)..(1370)
aaactacctaatcacccaaacgaagaaccaaacattttgaaattttttgggaaaattacaaagcacacgaatttagcaaaaagatccagttattaggtggaagcagattttgtagaaaaatggatggtagtatgaatttggggaatgagccaccaggagatggtggtggaggtggagggttgactagacaaggttcgatatactcgttgacgtttgatgagtttcagagcagtgtagggaaagattttgggtcaatgaacatggatgagttgttaaagaatatatggagtgctgaagaaacacaagccatggctagtggtgtggttccagttcttggtggaggtcaagagggtttgcagctgcagaggcaaggctcgttgactctgcctcgaacgcttagtcagaagacggttgatcaagtttggaaagatctatccaaagttggaagtagtggagtagggggaagtaacttgtctcaggtggctcaggctcagagtcagagtcagagtcagaggcagcaaacattaggtgaagtaactttggaggagtttttggttcgtgctggtgttgtgagagaggaagctcaggttgctgcaagagctcagattgctgagaacaataaaggcggttactttggtaatgatgccaacacaggtttctctgtcgagtttcagcagccttctccacgagttgttgccgctggtgtaatgggaaatcttggtgcagagactgcaaattctttgcaggttcaaggttctagtttgcctctgaatgtgaatggagctagaacaacataccagcaatcgcaacagcaacagccaatcatgcctaagcagcctggttttggttatggaacacaaatgggtcagcttaatagtcctgggataagaggtggtggtcttgtgggacttggagatcagtctttaacgaacaatgtgggctttgtccaaggtgcttctgctgcaattcctggagctttaggcgttggtgctgtgtcgcctgttacgccattgtcatcagaagggatagggaagagtaatggtgattcttcatcactctctccgtctccttacatgtttaatggtggtgtgagaggtagaaagagtggcactgtggagaaagttgtagagagaaggcaaaggagaatgataaagaaccgagaatcagctgcaaggtcccgggccaggaaacaggcttacaccgtggagcttgaagctgaagttgcaaagttaaaggaagagaatgacgagttacaacgaaagcaggcaaggatcatggaaatgcaaaagaatcaggagacggagatgaggaatcttctgcaaggaggtccaaagaaaaagctgaggaggacagagtcgggaccttggtgaatcaatcaatgccatcatacttagtttctgtagataaatgacatcccacttaggtgttttagttgaattagacttaatagagaagagctttcatcgtttatattgtaagctctctccatatatgttatgttttttacatacacaggatcatcagaatctcttttgctttatttagaccaagaattttgtgtgtgtttctcgttgttgtttgtcgttgtcgctattaaacctcaaaatgtactttcttgatcttggagttaccaattttgaagaattgaagtgttgtttggtt

SEQ ID NO: 2 - protein, 416 aa, Arabidopsis thaliana
MDGSMNLGNEPPGDGGGGGGLTRQGSIYSLTFDEFQSSVGKDFGSMNMDELLKNIWSAEETQAMASGVVPVLGGGQEGLQLQRQGSLTLPRTLSQKTVDQVWKDLSKVGSSGVGGSNLSQVAQAQSQSQSQRQQTLGEVTLEEFLVRAGVVREEAQVAARAQIAENNKGGYFGNDANTGFSVEFQQPSPRVVAAGVMGNLGAETANSLQVQGSSLPLNVNGARTTYQQSQQQQPIMPKQPGFGYGTQMGQLNSPGIRGGGLVGLGDQSLTNNVGFVQGASAAIPGALGVGAVSPVTPLSSEGIGKSNGDSSSLSPSPYMFNGGVRGRKSGTVEKVVERRQRRMIKNRESAARSRARKQAYTVELEAEVAKLKEENDELQRKQARIMEMQKNQETEMRNLLQGGPKKKLRRTESGPW

SEQ ID NO: 3 - DNA, 22 bp, Artificial Sequence
  Description of Artificial Sequence Primer
gcgtggaccgcttgctgcaact

SEQ ID NO: 4 - DNA, 21 bp, Artificial Sequence
  Description of Artificial Sequence Primer
tcaagctccacggtgtaagcc

SEQ ID NO: 5 - DNA, 33 bp, Artificial Sequence
  Description of Artificial Sequence Primer
ggggcggccgcatgacacaagccatggctagtg

SEQ ID NO: 6 - DNA, 31 bp, Artificial Sequence
  Description of Artificial Sequence Primer
gcagaagcaccttgacttccccctactccac

SEQ ID NO: 7 - DNA, 29 bp, Artificial Sequence
  Description of Artificial Sequence Primer
gtagggggaagtcaaggtgcttctgctgc

SEQ ID NO: 8 - DNA, 29 bp, Artificial Sequence
  Description of Artificial Sequence Primer
ggggagctctcaccaaggtcccgactctg

SEQ ID NO: 9 - DNA, 1828 bp, Oryza sativa
  CDS (218)..(1291)
ggtccggtttcctctcctcgcctcgccgcggcgcccgcgtcctcctcaccaacgccagagctccacatcccacctctcctcaggttcccgtctctgatccctcgttgcgttacggggaagcaggcgtggtggttgctccccgtgcggagttttcgattggtttggttgggtgagtttggtgctggggtgttggggattggttggttggagtttggagatggattttccgggagggagcgggaggcagcagcagctgccgccgatgacgccgctgccgttggcgaggcaggggtcggtgtactcgctcacgttcgacgagttccagagcacgctgggcggggtcgggaaggacttcgggtcgatgaacatggacgagctcctccgcagcatctggacggccgaggagtcgcacgccgtcggcgccgccacgacgacgacggcgacgacggcgtccgtggcggcggcggagcacgcggcggtgggggcgccgcccgttcagaggcaggggtcgctgaccctcccccgcacgctcagccagaagaccgtcgacgaggtctggcgcgacatgatgtgcttcggtggcggcggcgcctccaccgcgccggccgccgcggagcccccgccgccggcgcaccggcagcagacgctcggggagatcacgctggaggagttcctcgtgcgggccggcgtggtgagggaggacatgtcggtcccgcccgtcccgccggcgccgactcctacggcggctgctgtacctcccccgccgccgccgcagcagcagacgccgatgttgttcggtcagagcaatgtgttccctccgatggtgcctccgctctcgctgggaaatgggctggtctcgggagctgtcggacacggcggtggtggtgccgcgtcgttggtttcgccggtgaggccggtctcgtccaatggcttcggcaagatggaaggcggggacctgtcgtcgctgtcgccatcgccggtgccgtacgttttcaaaggtgggctgaggggaaggaaggcaccgggcatcgagaaggttgtcgagagaagacagcggcggatgatcaagaacagggagtctgccgcgaggtcgcgccagaggaaacaggcatatatgatggaattggaagctgaggtagcaaaacttaaggagctgaacgatgaactccagaaaaagcaggatgaaatgttggagcagcaaaagaatgaggttctagagagaatgagccgacaagttggaccgacagcaaagagaatttgccttcggaggactctgacgggtccatggtgatcgaagctaaaggcctagttacctgtaactagaacacagtcctttcaatgtgagcagtgatcatagtagttgtaggatggtgaagctgaagacctggttacctgtaactagaacacagtcctttcaaagtgagcagtgattacagtagtagtaggatggtgaagctgccagttacctgtaactagaacacagtcctttcaaagtgagtatagtagtaggttagttcagaatcgccagatcgatctgcccttgtaggccaaccattagtccgtgggcttgtctcttgggcggacaggctgcctgagatattcactcgataggttttagttgtctgagttatatccaagcattgtatgtaacctatttaaccatggtgcctgctacctgtattgtattttgctgctgtaggaacctatgtcggtgttaccatcttgttcttaatttgtgtacttctcttgccattttggataagatgtatctgaacttatacatatgtacttttccttgccattttggacaagatgtatctgaatttac

SEQ ID NO: 10 - protein, 357 aa, Oryza sativa
MDFPGGSGRQQQLPPMTPLPLARQGSVYSLTFDEFQSTLGGVGKDFGSMNMDELLRSIWTAEESHAVGAATTTTATTASVAAAEHAAVGAPPVQRQGSLTLPRTLSQKTVDEVWRDMMCFGGGGASTAPAAAEPPPPAHRQQTLGEITLEEFLVRAGVVREDMSVPPVPPAPTPTAAAVPPPPPPQQQTPMLFGQSNVFPPMVPPLSLGNGLVSGAVGHGGGGAASLVSPVRPVSSNGFGKMEGGDLSSLSPSPVPYVFKGGLRGRKAPGIEKVVERRQRRMIKNRESAARSRQRKQAYMMELEAEVAKLKELNDELQKKQDEMLEQQKNEVLERMSRQVGPTAKRICLRRTLTGPW